(19) United States
(12) Patent Application Publication
(10) Pub. No.: US 2005/0288351 A1
(43) Pub. Date: Dec. 29, 2005

(54) Azaspiro compounds for the treatment of pain

(75) Inventors: Claus Meese, Monheim (DE); Norma Selve, Troisdorf (DE); Dirk Schmidt, Duesseldorf (DE)

Correspondence Address: EDWARDS & ANGELL, LLP, P.O. BOX 55874, BOSTON, MA, 02205, US

(73) Assignee: Schwarz Pharma AG, Monheim Am Rhein (DE)

(21) Appl. No.: 10/936,134

(22) Filed: Sep. 7, 2004

Related U.S. Application Data
(63) Continuation of application No. PCT/EP03/01986, filed on Jan. 27, 2003.

(30) Foreign Application Priority Data
Mar. 7, 2002 (DE) 102 10 190.6

(51) Int. Cl. C07D 209/54; A61K 31/403

(52) U.S. Cl. 514/409; 548/408

(57) ABSTRACT

This invention relates to azaspiro compounds and their use as medications especially for the treatment of chronic, chronic-phlogistic and/or neuropathic pain. A compound that lends itself particularly well to the production of analgesics is 2-azaspiro[4.6]undecane-3-thion.

Neuropathic pain is a difficult-to-treat form of chronic pain that is caused by injuries or disorders of the peripheral and/or central nervous system and does not respond well to traditional analgesics.

In recent times, given the similarities in the pathophysiology of epilepsy and neuropathic pain, neuropathic pain has increasingly been treated with anticonvulsive agents. One example of these is Gabapentin which, while having been approved as an antiepileptic for some time, has lately gained augmented significance in the treatment of neuropathic pain (Tremont-Lukats in Drugs 60, 2000, 1029; Bock in “Nervenarzt” 72, 2001, 69).

While the way gabapentin works is not as yet fully understood, gabapentin's influence on the glutaminergic/GABAergic transmission and its effect on calcium channels offers a wide effective spectrum of activity that ranges from the treatment of epilepsy to neuropathic and other painful conditions such as migraine (Block in Nervenarzt 72, 2001, 69) or muscle and-skeletal pain (EP 1 047 414) and all the way to the treatment of depression (EP 552 240), neurodegenerative illnesses (EP 446 570), anxiety and panic conditions (EP 804 182) or mania (EP 825 857).

One drawback of gabapentin is that, when stored, it forms toxic gabapentin-lactam (2-azaspiro[4.5]decan-3-on), and that it is difficult to produce stable gabapentin formulations.

WO 99/25683 proposes a large number of pyrrolidinone compounds, substituted in position 4 and also encompassing azaspiro compounds such as gabapentin-lactam, for the treatment of diseases that are accompanied by elevated glutamate levels, for instance epilepsy, Alzheimer's, ALS or Parkinson's. An in-vitro model shows the effectiveness of gabapentin-lactam in ischemia and its reduction of the glutamate level. It also demonstrates the neuroprotective effect of gabapentin-lactam in a rat model. But because of its toxicity, gabapentin-lactam is not suitable for human therapy. Nor does WO 99/25683 give any indication to the effect that the pyrrolidones claimed are suitable for the treatment of neuropathic pain.

DE 25 57 220 describes N-substituted gabapentin-lactam derivatives for the treatment of epilepsy and cerebral disorders. There is no mention of its use as an analgesic.

Azaspiro compounds with aryl substituents for pain therapy are described in EP 337 547, EP 687 268, EP 880 528, EP 894 497, EP 906 315, EP 912 579, EP 929 554, EP 977 758 and EP 989 987. None of these documents suggests that desaryl azaspiro compounds also have analgetic potential.

EP A 116 347 proposes amino-substituted 1-azaspiro[4.5.]decanes and undecanes for the treatment of pain. It does not reveal any 2-azaspiro compounds or 1.3-diazaspiro compounds.

EP 310 321 describes 2-azaspiro compounds in which the aza atom is substituted with a nitrogenous side chain. It refers to these compounds as being immunomodulatory. No analgesic effect is mentioned.

In clinical practice there are but few agents that have proved effective and suitable for the treatment of chronic or neuropathic pain; accordingly, for that indication there remains a great need for innovative medications.

It is therefore the objective of this invention to introduce alternative medicines for the treatment of pain and especially of chronic, chronic-phlogistic and/or neuropathic pain.

A surprising discovery has revealed that gabapentin-lactam-derived azaspiro compounds of the general formula I offer greater analgesic potency than gabapentin and gabapentin-lactam while at the same time being less toxic than gabapentin-lactam.

The azaspiro compounds according to this invention that are suitable for therapeutic application are expressed in Formula I. 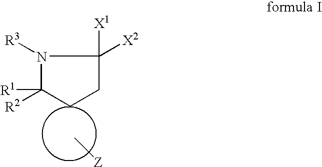
where

both X¹ and X² are hydrogen or jointly represent a thioxo or oximo group;

R¹ is hydrogen and R² is selected from among hydrogen, hydroxy, formyl, carboxy, halogen, mercapto, sulfonyl, amino, amido or from a —R⁷Q¹ group or where R¹ and R² jointly form an oxo- or thioxo group;

R³ represents hydrogen, hydroxy, amino or a —R⁸Q² group;

Z is a saturated or unsaturated ring that is connected to the first, heterocyclic ring via a joint C atom, that has 4-10 members including the azaspiro atom, that may have, in addition to carbon atoms, one or two ring-forming hetero atoms selected from O or S, and that is either unsubstituted or substituted with one or several substituents selected from among hydrogen, hydroxy, formyl, carboxy, halogen, mercapto, sulfonyl, amino, amido or a —R⁹Q³ group;

R⁷ and R⁹, independently of each other, represent C₁₋₅ alkyl, C₃-C₆ cycloalkyl, C₂₋₅ alkenyl, C₂₋₅ alkinyl, C₁₋₅ alkoxy, C₁₋₅ alkylcarbonyl, C₁₋₅ alkoxy carbonyl, C₁₋₅ alkylthio, C₁₋₅ alkylamino, C₁₋₅ alkyl sulfinyl, C₁₋₅ alkyl sulfonyl, C₁₋₅ alkylamino—C₁₋₅ alkyl, C₁₋₅ alkylthio-C₁₋₅ alkyl or C₁₋₅ alkoxy-C₁₋₅ alkyl;

R⁸ is selected from among C₁₋₅ alkyl, C₃₋₆ cycloalkyl, C₁₋₅ alkoxy, C₁₋₅ alkylcarbonyl, C₁₋₅ alkoxycarbonyl, C₁₋₅ alkylthio, C₁₋₅ alkylamino, C₁₋₅ alkylsulfinyl, C₁₋₅ alkylsulfonyl, C₁₋₅ alkylthio-C₁₋₅ alkyl or C₁₋₅ alkoxy-C₁₋₅ alkyl;

Q¹ and Q³, independently of each other, represent hydrogen, hydroxy, formyl, carboxy, halogen, mercapto, sulfonyl, amino or amido;

Q² is selected from among hydrogen, hydroxy, formyl, carboxy, halogen, mercapto, sulfonyl or amido.

The azaspiro compounds may be in the form of a free base or of pharmaceutically acceptable salts and in either form they are an object of this invention.

Moreover, depending on substituents, the azaspiro compounds may be obtained in various tautomeric forms which, if necessary, can be stabilized through salification. These tautomers and their salts as well are an object of this invention.

Pharmaceutically acceptable salts include all biocompatible salts that largely preserve the pharmacological properties of the active ingredients without causing any undesirable toxic effects. Examples include in particular the additive salts of inorganic or organic acids such as hydrogen chloride, hydrogen bromide, acetic acid, citric acid, tartaric acid, oxalic acid, fumaric acid, malic acid, succinic acid or methane sulfonic acid.

Also, as those skilled in the art are aware, azaspiro compounds may exist in optically inactive or active form depending on the substituents. Therefore, pure enantiomers as well as racemates or optically inactive compounds are explicitly included as objects of this invention.

For the purpose of this invention, the terms used above are to be understood as follows:

In this patent application, “C₁₋₅ alkyl” refers to a radical of a saturated aliphatic hydrocarbon group with 1-5 C-atoms that may or may not be branched. Examples of C₁₋₅ alkyls include methyl, ethyl, n-propyl, iso-propyl, n-butyl, iso-butyl, s-butyl, t-butyl, n-pentyl, iso-pentyl, neopentyl, t-pentyl, 1-methylbutyl, 2-methylbutyl, 1-ethylpropyl, 1.2-dimethylpropyl.

“C₂₋₅ alkenyl” and “C₂₋₅ alkinyl” in this patent application refer to radicals with 2-5 atoms that differ from the above-defined alkyls by virtue of at least one double or triple bond.

“C₃₋₆ cycloalkyl” refers to a radical of the group encompassing cyclopropyl, cyclobutyl, cyclopentyl and cyclohexyl.

For the purpose of this patent application, the term “hydrocarbon ring” refers to a substituted or unsubstituted ring whose ring-forming atoms consist exclusively of carbons and which is thus free of any ring-forming heteroatoms.

The term “C₁₋₅ alkoxy” refers to the radical —O—C₁₋₅ alkyl.

The term “C₁₋₅ alkythio” refers to the radical —S—C₁₋₅ alkyl.

The term “C₁₋₅ alkylamino” refers to the radical —NH—C₁₋₅ alkyl.

The term “C₁₋₅ alkylsulfinyl” refers to the radical —S(O)—C₁₋₅ alkyl.

The term “C₁₋₅ alkylsulfonyl” refers to the radical —S(O₂)—C₁₋₅ alkyl.

The term “C₁₋₅ alkylcarbonyl” refers to the radical —C(O)—C₁₋₅ alkyl.

The term “C₁₋₅ alkoxycarbonyl” refers to the radical —C(O)—O—C₁₋₅ alkyl.

The term “C₁₋₅ alkylamino-C₁₋₅ alkyl” refers to the C₁₋₅ alkyl-NH—C₁₋₅ alkyl group.

The term “C₁₋₅ alkythio-C₁₋₅ alkyl” refers to the C₁₋₅ alkyl-S—C₁₋₅ alkyl group.

The term “C₁₋₅ alkoxy-C₁₋₅ alkyl” refers to the C₁₋₅ alkyl-O—C₁₋₅ alkyl group.

The term “halogen” refers to a radical of the group including F, Cl, Br, I.

The term “thioxo group” refers to the ═S group.

One preferred object of this invention encompasses compounds per general formula I for medical applications, in which the ring Z including the azaspiro atom is a 5-8-member ring and, most desirably, a 5-, 6- or 7-member ring.

Another preferred object of the invention is a compound per general formula I in which the ring Z is a hydrocarbon ring. In another implementation of the invention the ring Z encompasses a ring-forming oxygen or sulfur atom.

In another preferred form of implementation of the invention, the ring Z is an unsubstituted ring and most desirably an unsubstituted hydrocarbon ring which, including the azaspiro atom, features 5, 6 or 7 ring-forming C-atoms.

In another form of implementation of the invention, the ring Z is a 5-, 6- or 7-member hydrocarbon ring substituted with a radical that is preferably selected from among hydroxy, formyl, carboxy, halogen, mercapto, sulfonyl, amino, amido or from the —R⁹Q³ group, where R⁹ is preferably a C₁₋₅ alkyl and Q³ is preferably selected from hydrogen, hydroxy, halogen, amine or sulfonate.

In another preferred form of implementation the ring Z is saturated and preferably constitutes a saturated hydrocarbon ring that is most desirably unsubstituted.

In a particularly preferred form of implementation of the invention the ring Z, including the spiro atom, consists of cyclopentane, cyclohexane or cycloheptane.

In another preferred form of implementation of the invention, both the substituents R¹ and R² of the compounds per formula I consist of hydrogen or together form an oxo or thioxo group.

In another preferred form of implementation of the invention the substituent R³ is C₁₋₅ alkylcarbonyl, for instance methyl carbonyl, or hydrogen.

In a particularly preferred implementation both the substituents R¹ and R² are hydrogen and R³ is hydrogen or methyl carbonyl.

Another preferred implementation of the invention relates to azaspiro compounds per general formula I, where

both X¹ and X² are hydrogen or jointly form a thioxo or oximo group;

R³ is hydrogen and R² is selected from among hydrogen, hydroxy, formyl, carboxy, halogen, mercapto, sulfonyl, amino, amido or from a —R⁷Q¹ group where R¹ and R² jointly form an oxo or thioxo group, with both R¹ and R² most desirably being hydrogen;

R³ is hydrogen or methyl carbonyl;

Z is a saturated or unsaturated hydrocarbon ring that is connected to the first, heterocyclic ring via a common C-atom and, including the azaspiro atom, consists of 5-7 members, is free of ring-forming heteroatoms and is unsubstituted, or substituted with one or two substituents selected from among hydroxy, formyl, carboxy, halogen, mercapto, sulfonyl, amino, amido or a —R⁹Q³ group where Z is ideally saturated;

R⁷ and R⁹, independently of each other, are C₁₋₅ alkyl, C₃-C₆ cycloalkyl, C₂₋₅ alkenyl, C₂₋₅ alkinyl, C₁₋₅ alkoxy, C₁₋₅ alkylcarbonyl, C₁₋₅ alkoxycarbonyl, C₁₋₅ alkylthio, C₁₋₅ alkylamino, C₁₋₅ alkylsulfinyl, C₁₋₅ alkylsulfonyl, C₁₋₅ alkylamino—C₁₋₅ alkyl, C₁₋₅ alkylthio-C₁₋₅ alkyl or C₁₋₅ alkoxy—C₁₋₅ alkyl;

Q¹ and Q³, independently of each other, are hydrogen, hydroxy, formyl, carboxy, halogen, mercapto, sulfonyl, amino or amido;

and where

the azaspiro compounds may be obtained as a free base or as pharmaceutically acceptable salts.

Azaspiro compounds per formula I that are particularly preferred for medicinal use are
2-azaspiro[4.5]decane
2-azaspiro[4.5]decane-3-thion
2-azaspiro[4.4]nonane-3-thion
2-azaspiro[4.6]undecane-3-thion
N-(2-azaspiro[4.5]decane-3)-oxim
N-(2-azaspiro[4.6]undecane)-3-oxim
1 -(2-azaspiro[4.5]dec-2yl)-ethanon

Another object of this invention encompasses pharmaceutical compositions containing an azaspiro compound per general formula I as described above, as well as at least one pharmaceutically acceptable adjuvant.

Those skilled in the art are aware of the fact that the pharmaceutical formulation can vary as a function of the intended mode of application. Accordingly, the pharmaceutical formulation may be adapted for instance for intravenous, intramuscular, intracutaneous, subcutaneous, oral, buccal, sublingual, nasal, transdermal, inhalational, rectal or intraperitoneal administration.

Again, those skilled in the art of pharmaceutics are familiar with suitable pharmaceutical carrier substances and adjuvants such as fillers, diffusers, binders, lubricants, stabilizers, flavorings, antioxidants, preservatives, dispersants or solvents, buffers or electrolytes, described for instance in such standard publications as Sucker, Fuchs und Speiser (“Pharmazeutische Technologie”, Georg Thieme Verlag, Stuttgart).

In a preferred implementation of the invention, the pharmaceutical compositions containing the novel compounds are administered orally and may be provided for instance in the form of capsules, tablets, powder, granules, lozenges or liquids.

Alternative pharmaceutical preparations may be in the form for instance of solutions for infusion or injection, of oils, suppositories, aerosols, sprays, microcapsules or microparticles.

The medicine may be a quick-release formulation whenever a fast-acting drug is needed, for instance in cases of acute chronic or neuropathic pain. Corresponding formulations are described in such publications as EP 159 237 or EP 1 126 821.

If, on the other hand, protracted release is desired, a slow-acting formulation can be made available. Prior art has produced corresponding oral formulations.

The pharmaceutical formulations preferably include a compound per general formula I 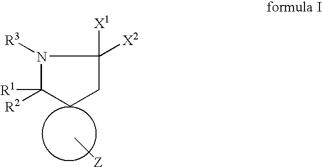
where the nature of R¹, R², R³, X¹, X² and Z is as indicated above.

A preferred object of this invention encompasses pharmaceutical compositions containing azaspiro compounds per general formula I, in which the ring Z of the azaspiro compound is an unsubstituted ring and/or a saturated hydrocarbon ring preferably with 5, 6 or 7 ring-forming C-atoms including the azaspiro atom.

In a particularly preferred implementation of the invention, the pharmaceutical formulation encompasses an azaspiro compound per general formula I in which the ring Z, including the spiro atom, is a saturated, unsubstituted hydrocarbon ring especially of cyclopentane, cyclohexane or cycloheptane.

In another preferred implementation of the invention, the pharmaceutical formulation encompasses an azaspiro compound per general formula I, where the substituents R¹ and R² are both hydrogen or jointly form an oxo or thioxo group.

In a particularly preferred implementation both the substituents R¹ and R² are hydrogen and R³ is either hydrogen or a methyl carbonyl group.

In another preferred implementation of the invention, X¹ and X² jointly form a thioxo or oximo group, both R¹ and R² are hydrogen, R³ is hydrogen or methyl carbonyl, and Z is a saturated hydrocarbon ring that is most preferably unsubstituted and preferably contains 5-8 ring-forming atoms.

Particularly preferred pharmaceutical compositions encompass azaspiro compounds per formula I selected from among
2-azaspiro[4.5]decane-3-thion
2-azaspiro[4.4]nonane-3-thion
2-azaspiro[4.6]undecane-3-thion
2-azaspiro[4.5]decane
N-(2-azaspiro[4.5]decane-3)-oxim
N-(2-azaspiro[4.6]undecane)-3-oxim
1-(2-azaspiro[4.5]dec-2yl)-ethanon
   and their pharmaceutically acceptable salts as well as a pharmaceutically acceptable carrier substance or adjuvant.

Another object of this invention relates to retail packages containing at least one pharmaceutical formulation as described above as well as instructions for its use. A retail package of this type may contain other medications as well. For example, the retail package could additionally contain another analgesic, a sedative, an ergotamine derivative, an antiemetic agent, an anti-inflammatory agent or an antidepressant.

Surprisingly, in pharmacologic comparison studies the compounds according to this invention have displayed a high level of effectiveness in a formalin test, an in-vivo test for the predictability of the potential effectiveness of a substance in the treatment of chronic or chronic-phlogistic and/or neuropathic pain (Tjolsen and Herle, Handbook Exp. Pharmacol. Vol 130, Ed: Dickenson & Besson, Springer Verlag 1997, page 6).

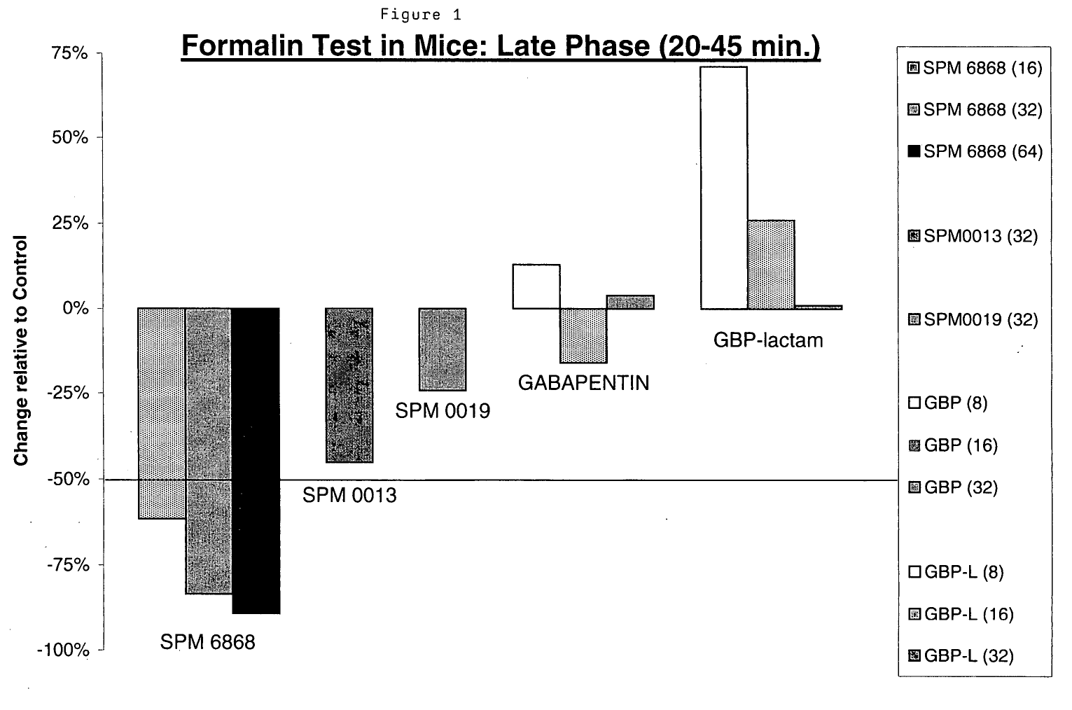

FIG. 1 and Table 1 show the reaction of test animals (10 mice each) 20-45 minutes after the intraperitoneal administration of selected compounds. In each case, the maximum dosage selected for the concentration of azaspiro compounds was held, by a factor of about 2, below the toxic dose previously determined in the IRWIN test (Irwin, Psychopharmacologia 13 (1968) 222).

The abbreviations used in FIG. 1 signify the following: SPM 6868 stands for 2-azaspiro[4.6]undecane-3-thion, SPM 0013 stands for 2-azaspiro[4.5]decane-3-thion, SPM 0019 stands for 2-azaspiro[4.5]decane. GBP means gabapentine. GBP-L represents gabapentin-lactam. Parenthesized in the legend behind the name of the substance is the dosage of the substance in mg/kg of body weight.

As can be seen in FIG. 1, the compounds per this invention surprisingly exhibited a significantly greater potency in the formalin test than gabapentin and gabapentin-lactam that were used for comparison.

Moreover, after 30-45 minutes in the formalin test referred to above the two compounds N-(2-azaspiro[4.5]decane-3)-oxim (SPM 6850) and N-(2-azaspiro[4.6]undecane-3)-oxim (SPM 6873) revealed an average reduction of the pain reaction by 44% and 36.5%, respectively, as shown in Table 1:

TABLE 1

| | | Mean deviation of the pain reaction | | |
|---|---|---|---|---|
| | | compared to control (%) | | |
| | Dosage | (n minutes after administration of formalin) | | |
| Compound | (mg/kg) | 20-25′ | 30-35′ | 40-45′ |
| SPM 6868 | | | | |
| Test series 1 | 64 | −89 | −67 | −83 |
| (n = 10) | 32 | −83 | −0 | −24 |
| | 16 | −62 | −29 | −49 |
| Test series 2 | 64 | −100 | −98 | −93 |
| (n = 10) | 16 | −42 | −30 | −31 |
| | 4 | −53 | −7 | −16 |
| SPM 0013 | 32 | −99 | −32 | −4 |
| (n = 10) | | | | |
| SPM 0019 | 32 | −67 | −21 | −26 |
| (n = 10) | | | | |
| SPM 6850 | 32 | (+) | −50 | −38 |
| (n = 10) | | | | |
| SPM 6873 | 16 | (+) | −35 | −38 |
| (n = 10) | | | | |
| GBP-L | | | | |
| Test series 1 | 32 | −64 | −15 | −46 |
| (n = 10) | 16 | −74 | −40 | −41 |
| | 8 | −58 | −18 | −15 |
| Test series 2 | 32 | (+) | −10 | n/d |
| (n = 10) | 16 | (+) | (+) | n/d |
| | 8 | (+) | (+) | n/d |
| Morphine* | 8 | −87 | −95 | −88 |

(+): Intensified pain reaction
*Average of 9 test series
n/d = not determined

Finally, the side effects of the compounds per this invention that set in above the maximum tolerable dosages (sedation, tremor, hypothermia) proved to be substantially less severe than those of the GPL where a significant lethality rate was observed.

It follows that the azaspiro compounds that are suitable for therapy lend themselves particularly well to the treatment of pain, especially chronic, chronic-phlogistic and/or neuropathic pain.

Accordingly, one object of this invention is the use of an azaspiro compound per general formula II 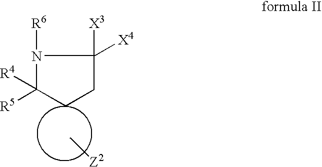
where

both X³ and X⁴ are either hydrogen or they jointly represent a thioxo or an oximo group;

R⁴ is hydrogen and R⁵ is selected from among hydroxy, formyl, carboxy, halogen, mercapto, sulfonyl, amino, amido or a —R¹⁰Q⁴ group or where R⁴ and R⁵ jointly form an oxo or thioxo group;

R⁶ is hydrogen, hydroxy, amino or a —R¹¹Q⁵ group;

Z² is a saturated or unsaturated ring that is connected to the first, heterocyclic ring via a common C-atom, that consists of 4-10 members including the azaspiro atom, that may have, in addition to carbon atoms, one or two ring-forming heteroatoms selected from N, O or S, and that is unsubstituted or substituted with one or several substituents selected from among hydrogen, hydroxy, formyl,.carboxy, halogen, mercapto, sulfonyl, amino, amido or a —R¹²Q⁶ group;

R¹⁰, R¹¹ and R¹², independently of one another, represent C₁₋₅ alkyl, C₃-C₆ cycloalkyl, C₂₋₅ alkenyl, C₂₋₅ alkinyl, C₁₋₅ alkoxy, C₁₋₅ alkylcarbonyl, C₁₋₅alkoxycarbonyl, C₁₋₅ alkylthio, C₁₋₅ alkylamino, C₁₋₅ alkylsulfinyl, C₁₋₅ alkylsulfonyl, C₁₋₅ alkylamino-C₁₋₅ alkyl, C₁₋₅ alkylthio-C₁₋₅ alkyl or C₁₋₅ alkoxy-C₁₋₅ alkyl;

Q⁴, Q⁵ and Q⁶, independently of one another, are hydrogen, hydroxy, formyl, carboxy, halogen, mercapto, sulfonyl, amino or amido;

as well as possible tautomers and/or pharmaceutically acceptable salts

for producing a medication for the treatment of pain, especially chronic, chronic-phlogistic and/or neuropathic pain.

The medications containing the azaspiro compounds per this invention can essentially be used for treating various types of pain such as migraine, skeletal and muscle pain, etc. However, analgesics containing these novel azaspiro compounds lend themselves particularly well to the treatment of chronic, chronic-phlogistic and/or neuropathic pain.

Neuropathic pain is a complex syndrome often encountered as a consequence of injuries, infections, metabolic disorders and degenerative diseases of the nervous system. Examples of a neuropathic pain syndrome include pseudesthesia, postherpetic neuralgia following herpes zoster, painful diabetic neuropathy, complex regional pain syndromes, various types of cancer-related pain, neuropathic pain in connection with multiple sclerosis or with injuries to a major neuroplexus, to the spinal cord or to the brainstem.

One preferred object of the invention is the use of the compounds per general formula II in analgesics where the ring Z², including the azaspiro atom, is a 5-8-member and especially a 5-, 6- or 7-member ring.

In another preferred implementation of the invention, the production of the analgesic employs a compound per general formula II in which the ring Z² is a hydrocarbon ring. In a variation of the invention, the ring Z² includes a ring-forming oxygen or sulfur atom.

In another preferred implementation of the invention, the ring Z² of the azaspiro compound that is used for producing the analgesic is an unsubstituted ring and most desirably an unsubstituted hydrocarbon ring with 5, 6 or 7 ring-forming C-atoms including the azaspiro atom.

In another implementation of the invention, the ring Z² is a 5-, 6- or 7-member hydrocarbon ring substituted with a radical that is preferably selected from among hydroxy, formyl, carboxy, halogen, mercapto, sulfonyl, amino, amido or the —R¹²Q⁶ group where R¹² preferably consists of C₁₋₅ alkyl and Q⁶ is preferably selected from among hydrogen, hydroxy, halogen, amino or sulfonyl.

In another preferred implementation, the ring Z² that is used for producing the azaspiro compound for the analgesics is saturated and preferably constitutes a saturated hydrocarbon ring which is ideally unsubstituted.

In a particularly preferred implementation of the invention, the ring Z² of the analgesic agent per formula II, including the spiro atom, consists of cyclopentane, cyclohexane or cycloheptane.

In another preferred implementation of the invention, the analgesic is produced with an azaspiro compound per general formula II in which both the substituents R⁴ and R⁵ are hydrogen or jointly form an oxo or thioxo group.

In another preferred implementation of the invention the substituent R⁶ is methyl carbonyl or hydrogen.

In a particularly preferred implementation, the substituents R⁴, R⁵ and R⁶ are all hydrogen.

In one form of implementation of the invention, the medication for treating pain, especially chronic, chronic-phlogistic and/or neuropathic pain, is produced with an azaspiro compound per general formula II, where

X³ and X⁴ are either both hydrogen or jointly form a thioxo or oximo group;

R⁴ is hydrogen and R⁵ is selected from among hydroxy, formyl, carboxy, halogen, mercapto, sulfonyl, amino, amido or a —R¹⁰Q⁴ group or where R⁴ and R⁵ jointly form an oxo or thioxo group, and where most preferably both R⁴ and R⁵ are hydrogen;

R⁶ is hydrogen or methyl carbonyl, with R⁶ ideally being hydrogen;

Z² is a saturated or unsaturated hydrocarbon ring that is connected to the first heterocyclic ring via a common C-atom, that consists of 5-7 members including the azaspiro atom, that is free of ring-forming heteroatoms, and that is unsubstituted or substituted with one or two substituents selected from among hydrogen, hydroxy, formyl, carboxy, halogen, mercapto, sulfonyl, amino, amido or a —R¹²Q⁶ group, and where, most preferably, Z² is a saturated hydrocarbon ring;

R¹⁰ and R¹², independently of each other, represent C₁₋₅ alkyl, C₃-C₆ cycloalkyl, C₂₋₅ alkenyl, C₂₋₅ alkinyl, C₁₋₅ alkoxy, C₁₋₅ alkylcarbonyl, C₁₋₅ alkoxycarbonyl, C₁₋₅ alkylthio, C₁₋₅ alkylamino, C₁₋₅ alkylsulfinyl, C₁₋₅ alkylsulfonyl, C₁₋₅ alkylamino-C₁₋₅ alkyl, C₁₋₅ alkylthio-C₁₋₅ alkyl or C₁₋₅ alkoxy-C₁₋₅ alkyl;

Q⁴ and Q⁶, independently of each other, are hydrogen, hydroxy, formyl, carboxy, halogen, mercapto, sulfonyl, amino or amido, and where the azaspiro compound may be in the form of a free base or of a pharmaceutically acceptable salt.

Particular preference for producing an analgesic is given to those azaspiro compounds which in the formalin test, described in Example #8, bring about a mean deviation of the pain reaction of at least—40% and preferably at least—50% or—60% in at least one and preferably in at least two of the test periods (20-25′, 30-35′, 40-45′ after the administration of formalin).

For the purpose of this patent application, the term “mean deviation of the pain reaction” refers to a relative deviation that is obtained when the average time of pain reaction of 10 animals treated with active agents in a test series as described in implementation Example #8 is compared, over a defined period (20-25′, 30-35′ or 40-45′ after formalin injection), with the average time of the pain reaction of 10 control animals treated with an excipient. A reduction in the pain reaction is expressed in negative percentage figures.

Especially preferred for producing a medication for treating pain, in particular chronic, chronic-phlogistic and/or neuropathic pain, are azaspiro compounds per formula II selected from among
2-azaspiro[4.5]decane-3-thion
2-azaspiro[4.4]nonane-3-thion
2-azaspiro[4.6]undecane-3-thion
2-azaspiro[4.5]decane
N-(2-azaspiro[4.5]decane-3)-oxim
N-(2-azaspiro[4.6]undecane-3)-oxim
1-(2-azaspiro[4.5]dec-2yl)-ethanon
   and their pharmaceutically acceptable salts.

Consequently, this invention relates to a new azaspiro compound selected from among
2-azaspiro[4.5]decane-3-thion
2-azaspiro[4.4]nonane-3-thion
2-azaspiro[4.6]undecane-3-thion
2-azaspiro[4.5]decane
N-(2-azaspiro[4.5]decane-3)-oxim
N-(2-azaspiro[4.6]undecane-3)-oxim
1-(2-azaspiro[4.5]dec-2yl)-ethanon

The following examples will explain this invention in more detail.

### 1. Production of 2-azaspiro[4.5]decane-3-thion

1.01 g (6.6 mmol) of gabapentin-lactam, 1.6 g (4.0 mmol) Lawson reagent and 20 ml toluene were placed in a single-neck flask. The yellow, recirculating solution (at 130° C.) was agitated for 20 hours. Once cooled, it was filtered via a silica gel column (with acetic acid as the eluent). The solvent was siphoned off.

The solid yellow substance was repurified via a silica gel column, followed by another column separation with aluminum oxide. The solvent was again siphoned off and the residue was recrystallized from 40 ml diisopropyl ether at 4° C.

The yield was 25% of theoretical (278 mg)

The melting point was 121.9° C. (Büchi B-545, 1° C./min)

NMR (CDCl₃): 204.64; 60.19; 55.44; 42.18; 36.03; 25.35; 22.90.

### 2. Production of 2-azaspiro[4.4]nonane-3-thion

3.93 g (28.2 mmol) of 2-azaspiro[4.4]nonane-3-on, 6.5 g Lawson reagent and 100 ml toluene were placed in a single-neck flask. The reaction product mixture was reflux-heated for 20 hours, resulting in a yellow solution. The solvent was removed using a rotary evaporator.

The residue was chromatographed via an aluminum oxide column (eluent: dichloromethane).

The eluent was removed by distillation and the remaining solids were boiled in 100 ml diisopropyl ether and 15 ml dichloromethane with activated charcoal. The activated charcoal was hot-filtered and the filtrate was cooled to +4° C., resulting in the precipitation of white crystals.

The yield was 1.14 g (26% of theoretical).

NMR (CDCl₃): 205.24; 60.50; 55.66; 49.42; 37.60; 23.73.

### 3. Production of 2-azaspiro[4.6]undecane-3-thion

4.18 g (25 mmol) of 2-azaspiro[4.6]undecane-3-on, 5.8 g (14.3 mmol) Lawson reagent and 125 ml toluene were placed in a single-neck flask, then reflux-recirculated for 18 hours. After cooling the solution was filtered out through aluminum oxide (neutral). The filtrate was turned over and the residue was recrystallized from 60 ml diisopropyl ether.

The yield was 1.2 g (26.2% of theoretical).

The melting point was 132.3° C. (Büchi B-545, 1° C./min)

NMR: 205.05; 61.59; 57.32; 45.83; 39.50; 28.68; 23.26.

### 4. Production of 2-azaspiro[4.5]decane

28 ml of 1.0 M lithium aluminum hydride in THF (28.0 mmil) was placed in a 100 ml triple-neck flask with magnetic agitator, drip funnel and reflux condenser. Added to this by drip-feeding, with agitation and cooling to 0° C.-10° C., was a solution of 5.01 g (32.7 mmol) gabapentin-lactam (2-aza-spiro[4.5]decane-3-on) in 30 ml THF. The reaction was highly exothermic with concurrent H₂ generation.

After completion of the drip-feed the substance was reflux-heated for another 4 hours.

The reaction product was subsequently cooled to 0° C. and mixed with a blend of 2 ml water and 2 ml THF so as to eliminate the surplus LiAlH₄. After final decomposition another 10 ml of THF was added to dilute the solution. The reaction product was mixed with 10 g NaSO₄ and agitated for another 10 minutes.

The sodium sulfate was filtered off and washed three times each with 20 ml THF. The solvent was siphoned off, leaving amine in the form of a clear, colorless oil.

The yield was 4.06 g (89.2% of theoretical).

NMR (CDCl₃): 59.03; 46.28; 42.98; 38.72; 37.01; 25.66; 23.78.

### 5. Production of N-(2-azaspiro[4.5]decane-3)-oxim

1.69 g of 2-azaspiro[4.5]decane-3-thion, produced as in Example #1, was dissolved in 200 ml ethanol. Added to this was 25 ml of 50% hydroxylamine solution in water and the preparation was then agitated overnight at room temperature.

The preparation was fully turned over. The oily residue was taken up in 10 ml water, crystallizing shortly thereafter. The product was siphoned off and dried in a vacuum at room temperature.

The yield of the colorless product was 63.10% of theoretical (1.06 g).

The melting point was determined at 156.9° C. (Büchi B-545, 1° C./min).

NMR (CDCl₃): 158.30; 55.59; 40.98; 38.86; 36.09; 25.93; 23.27.

### 6. Production of N-(2-azaspiro[4.6]undecane-3)-oxim

550 mg of 2-azaspiro[4.6]undecane-3-thion, produced as in Example #3, was dissolved in 60 ml ethanol. Added to this was 15 ml of 50% hydroxylamine solution in water and the preparation was then agitated overnight at room temperature.

The preparation was fully turned over. The oily residue was taken up in 10 ml water, crystallizing shortly thereafter. The product was siphoned off and dried in a vacuum at room temperature.

The yield of the colorless product was 91.1% of theoretical (460 mg).

The melting point was determined at 140.8° C. (Büchi B-545, 1° C./min).

NMR (CDCl₃): 158.38; 56.90; 44.36; 40.87; 39.01; 29.21; 23.61; 23.25.

### 7. Production of 1-(2-azaspiro[4.5]dec-2yl)-ethanon

1.98 g of 2-azaspiro[4.5]decane, produced as in Example #4, was diluted in 10 ml dichloromethane. Added to this in an argon atmosphere was 2.5 ml triethylamine and the solution was cooled to 0° C. A solution of 1.2 ml acetylchloride in 10 ml dichloromethane was added by drip-feeding while the solution was agitated, followed by agitation for 2 hours at 0° C. and one hour at room temperature.

20 ml water was added to the reaction mixture and the phases were separated. The organic yellow phase was rinsed with 20 ml of 1M HCl and 20 ml water. The aqueous phases were washed with dichloromethane. The organic phases were dried via sodium sulfate, the sodium sulfate was filtered out and the solvent was removed by distillation. The residue was chromatographed twice through a silica gel column (eluent: triethylamine/acetic ester 1:9).

The yield was 100% of theoretical (2.54 g).

NMR (CDCl₃): 169.28; 58.03; 55.49; 45.74; 43.87; 35.17; 25.98; 23.20; 22.01.

### 8. In-Vivo Test for determining the Analgesic Efficacy of the azaspiro Compounds

The test was conducted as described by Wheeler-Aceto (Psychopharmacology 104, 1991, 35).

NMRl mice having a weight of 20-25 g were kept under controlled conditions (22±2° C., 40-70% relative humidity). 25 μl of a 5% formocarbonyl solution was injected in the hind leg and the leg-licking frequency was then clocked for 5 minutes each at defined intervals (20, 30, 40 minutes).

First, the highest possible non-toxic concentration of the respective test substances that could be used was determined by an IRWIN test (Irwin, Psychopharmacologia 13, 1968, 222).

The test substances were dissolved in a physiological salt solution with 0.5% sodium carboxymethyl cellulose and were each measured in 1-3 dosages that had been applied intraperitoneally 10 minutes before administration of formalin. The comparative reference value was provided by a control excipient (10 ml/kg).

The test was performed in blind fashion on 10 mice per test series. The evaluation was based on a comparison of the treated animals with control excipients at three different times. To that end, a pain-reaction mean value per time period was established for the 10 animals of a test series followed by the determination of the relative deviation of the animals treated with the effective agent from the control animals in each of the three different periods. Accordingly, an average 50% reduction of the pain reaction for the treated animals is obtained when in a defined period (e.g. 30-35′ after the formalin injection) the leg-licking duration (averaged over the 10 test animals) is reduced by 50% compared to the untreated animals. The statistical significance was determined using the Mann-Whitney U-test.

1-23. (canceled)

24. A pharmaceutical composition comprising a compound of the following formula I 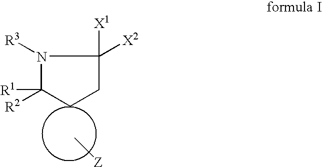
wherein
X¹ and X² jointly constitute a thioxo group;
R¹ is hydrogen and R² is selected from among hydrogen, hydroxy, formyl, carboxy, halogen, mercapto, sulfonyl, amino, amido or from a —R⁷Q¹ group or where R¹ and R² jointly form an oxo- or thioxo group;
R³ represents hydrogen, hydroxy, amino or a —R⁸Q² group;
Z is a saturated ring that is connected to the first, heterocyclic ring via a common C atom, that has 5-8 members including the azaspiro atom, that may have, in addition to carbon atoms, one or two ring-forming hetero atoms selected from O or S, and that is either unsubstituted or substituted with one or more substituents selected from among hydrogen, hydroxy, formyl, carboxy, halogen, mercapto, sulfonyl, amino, amido or a —R⁹Q³ group;
R⁷ and R⁹, independently from each other, represent C₁₋₅ alkyl, C₃-C₆ cycloalkyl, C₂₋₅ alkenyl, C₂₋₅ alkynyl, C₁₋₅ alkoxy, C₁₋₅ alkyl carbonyl, C₁₋₅ alkoxy carbonyl, C₁₋₅ alkylthio, C₁₋₅ alkylamino, C₁₋₅ alkyl sulfinyl, C₁₋₅ alkyl sulfonyl, C₁₋₅ alkylamino-C₁₋₅ alkyl, C₁₋₅ alkylthio-C₁₋₅ alkyl or C₁₋₅ alkoxy-C₁₋₅ alkyl;
R⁸ is selected from among C₁₋₅ alkyl, C₃-C₆ cycloalkyl, C₁₋₅ alkoxy, C₁₋₅ alkyl carbonyl, C₁₋₅ alkoxy carbonyl, C₁₋₅ alkylthio, C₁₋₅ alkylamino, C₁₋₅ alkyl sulfinyl, C₁₋₅ alkyl sulfonyl, C₁₋₅ alkylthio-C₁₋₅ alkyl or C₁₋₅ alkoxy-C₁₋₅ alkyl;
Q¹ and Q³, independently of each other, represent hydrogen, hydroxy, formyl, carboxy, halogen, mercapto, sulfonyl, amino or amido;
Q² is selected from among hydrogen, hydroxy, formyl, carboxy, halogen, mercapto, sulfonyl or amido;
or a tautomer and/or pharmaceutically acceptable salt thereof.

25. A composition of claim 24 wherein R³ is hydrogen or C₁₋₅ alkyl carbonyl.

26. A composition of claim 24 wherein Z is a 5-, 6- or 7-member ring.

27. A composition of claim 24 wherein Z is an unsubstituted ring.

28. A composition of claim 24 wherein Z is cyclopentane, cyclohexane or cycloheptane.

29. A composition of claim 24 wherein both R¹ and R² are hydrogen or jointly form an oxo group or a thioxo group.

30. A composition of claim 24 wherein both R¹ and R² are hydrogen and R³ is either hydrogen or methyl carbonyl.

31. A composition of claim 24 wherein the composition comprises one or more pharmaceutically acceptable adjuvants.

32. A pharmaceutical composition comprising a compound selected from among
2-azaspiro[4.5]decane-3-thion;
2-azaspiro[4.4]nonane-3-thion; or
2-azaspiro[4.6]undecane-3-thion;
or a pharmaceutically acceptable salt thereof.

33. A composition of claim 34 further comprising one or more pharmaceutical adjuvants.

34. A method for treating a patient suffering from or susceptible to pain, comprising administering to the patient an effective amount of a compound of the following formula II: 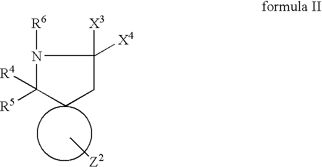
wherein
both X³ and X⁴ jointly constitute a thioxo group;
R⁴ and R⁵ independently represent hydrogen, hydroxy, formyl, carboxy, halogen, mercapto, sulfonyl, amino, amido or a —R¹⁰Q⁴ group or jointly form an oxo or thioxo group;
R⁶is hydrogen, hydroxy, amino or a —R¹¹Q⁵ group;
Z² is a saturated ring that is connected to the first, heterocyclic ring via a common C-atom, that consists of 4-10 members including the azaspiro atom, that may have, in addition to carbon atoms, one or two ring-forming heteroatoms selected from N, O or S, and that is either unsubstituted or substituted with one or more substituents selected from among hydrogen, hydroxy, formyl, carboxy, halogen, mercapto, sulfonyl, amino, amido or a —R¹²Q⁶ group;
R¹⁰, R¹¹ and R¹² independently represent C₁₋₅ alkyl, C₃-C₆ cycloalkyl, C₂₋₅ alkenyl, C₂₋₅ alkynyl, C₁₋₅ alkoxy, C₁₋₅ alkylcarbonyl, C₁₋₅ alkoxycarbonyl, C₁₋₅ alkylthio, C₁₋₅ alkylamino, C₁₋₅ alkylsulfinyl, C₁₋₅ alkylsulfonyl, C₁₋₅ alkylamino-C₁₋₅ alkyl, C₁₋₅ alkylthio-C₁₋₅ alkyl or C₁₋₅ alkoxy-C₁₋₅ alkyl;
Q⁴, Q⁵ and Q⁶ independently are hydrogen, hydroxy, formyl, carboxy, halogen, mercapto, sulfonyl, amino or amido;
or a tautomer and/or pharmaceutically acceptable salt thereof.

35. The method of claim 34 wherein R⁶ is hydrogen or C₁₋₅ alkyl carbonyl.

36. The method of claim 34 wherein Z² is a 5-, 6- or 7-member ring.

37. The method of claim 34 wherein Z² is an unsubstituted ring.

38. The method of claim 34 wherein Z² is cyclopentane, cyclohexane or cycloheptane.

39. The method of claim 34 wherein both R⁴ and R⁵ are hydrogen or jointly form an oxo or thioxo group.

40. The method of claim 34 wherein both R⁴ and R⁵ are hydrogen and R⁶ is hydrogen or methyl carbonyl.

41. The method of claim 34 where the compound is selected from among
2-azaspiro[4.5]decane-3-thion;
2-azaspiro[4.4]nonane-3-thion; or
2-azaspiro[4.6]undecane-3-thion;
or a pharmaceutically acceptable salt thereof.

42. The method of claim 34 wherein the pain is chronic pain, chronic-phlogistic pain and/or neuropathic pain.

43. The method of claim 34 wherein the patient is suffering from chronic pain.

44. The method of claim 34 wherein the patient is suffering from chronic-phlogistic pain.

45. The method of claim 34 wherein the patient is suffering from neuropathic pain.

46. The method of claim 34 wherein the patient is selected as suffering from pain and the compound is administered to the selected patient.

47. The method of claim 34 wherein the patient is selected as suffering from chronic pain, chronic-phlogistic pain and/or neuropathic pain and the compound is administered to the selected patient.

48. A compound selected from among
2-azaspiro[4.5]decane-3-thion;
2-azaspiro[4.4]nonane-3-thion; or
2-azaspiro[4.6]undecane-3-thion;
or a pharmaceutically acceptable salt thereof.
